(19) United States
(12) Patent Application Publication
(10) Pub. No.: US 2021/0038199 A1
(43) Pub. Date: Feb. 11, 2021

(54) METHODS AND APPARATUSES FOR DETECTING MOTION DURING COLLECTION OF ULTRASOUND DATA

(71) Applicant: Butterfly Network, Inc., Guilford, CT (US)

(72) Inventors: Abraham Neben, Guilford, CT (US); Kurt Guenther, Guilford, CT (US)

(73) Assignee: Butterfly Network, Inc., Guilford, CT (US)

(21) Appl. No.: 16/988,096

(22) Filed: Aug. 7, 2020

Related U.S. Application Data
(60) Provisional application No. 62/885,181, filed on Aug. 9, 2019.

(51) Int. Cl. A61B 8/00; A61B 8/08

(52) U.S. Cl. CPC A61B 8/54; A61B 8/4254; A61B 8/483

(57) ABSTRACT

Aspects of the technology described herein relate to detecting motion during collection of ultrasound data. Some embodiments include automatically comparing first ultrasound data collected from a set of locations at a first time and second ultrasound data collected from the set of locations at a second time to determine that a difference between the first and second ultrasound data exceeds a threshold difference. The set of locations may be scanline. Some embodiments include determining based on motion data from a motion sensor on the ultrasound device that an amount of motion exceeds a threshold amount of motion. An action may be performed based on determining that the difference between the first and second ultrasound data exceeds the threshold difference and/or that the amount of motion exceeds the threshold amount of motion. For example, the ultrasound data collection may be aborted, restarted, or a notification about motion may be generated.

### CROSS-REFERENCE TO RELATED APPLICATIONS

This application claims the benefit under 35 U.S.C. § 119(e) of U.S. Patent Application Ser. No. 62/885,181, filed Aug. 9, 2019 under Attorney Docket No. B1348.70150US00, and entitled “METHODS AND APPARATUSES FOR DETECTING MOTION DURING COLLECTION OF ULTRASOUND DATA,” which is hereby incorporated by reference herein in its entirety.

### FIELD

Generally, the aspects of the technology described herein relate to collection of ultrasound images. Certain aspects relate to detecting motion during collection of ultrasound data.

### BACKGROUND

Ultrasound devices may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, a method for detecting motion during a three-dimensional ultrasound imaging sweep includes automatically comparing, by a processing device in operative communication with an ultrasound device, first ultrasound data collected from a set of locations at a first time by the ultrasound device and second ultrasound data collected from the set of locations at a second time by the ultrasound device, wherein the first and second times are during the three-dimensional ultrasound imaging sweep.

In some embodiments, the set of locations comprises a scanline. In some embodiments, the ultrasound device is configured to remain substantially motionless during the three-dimensional ultrasound imaging sweep. In some embodiments, the set of locations is one set among multiple sets of location within a three-dimensional volume from which the ultrasound device collects ultrasound data during the three-dimensional ultrasound imaging sweep.

In some embodiments, automatically comparing the first and second ultrasound data to determine the difference between the first and second ultrasound data comprises computing a cross-correlation between the first and second ultrasound data. In some embodiments, automatically comparing the first and second ultrasound data to determine the difference between the first and second ultrasound data comprises computing a maximum absolute difference between the first and second ultrasound data. In some embodiments, automatically comparing the first and second ultrasound data to determine the difference between the first and second ultrasound data comprises computing an average absolute difference between the first and second ultrasound data.

In some embodiments, the method further comprises automatically determining that the difference between the first and second ultrasound data exceeds a threshold difference. In some embodiments, automatically comparing the first and second ultrasound data to determine the difference between the first and second ultrasound data comprises computing a cross-correlation between the first and second ultrasound data and automatically determining that the difference between the first and second ultrasound data exceeds a threshold difference comprises determining that the cross-correlation is less than a certain value. In some embodiments, automatically comparing the first and second ultrasound data to determine the difference between the first and second ultrasound data comprises computing a maximum absolute difference between the first and second ultrasound data and automatically determining that the difference between the first and second ultrasound data exceeds a threshold difference comprises determining that the maximum absolute difference is greater than a certain value. In some embodiments, automatically comparing the first and second ultrasound data to determine the difference between the first and second ultrasound data comprises computing an average absolute difference between the first and second ultrasound data and automatically determining that the difference between the first and second ultrasound data exceeds a threshold difference comprises determining that the average absolute difference is greater than a certain value.

In some embodiments, the method further includes configuring the ultrasound device to collect the first ultrasound data from the set of locations at the first time and the second ultrasound data from the set of locations at the second time, and receiving the first and second ultrasound data. further comprising configuring the ultrasound device to collect ultrasound data from only one other set of location between the first time and the second time. In some embodiments, the method further includes configuring the ultrasound device to collect ultrasound data from multiple other set of locations between the first time and the second time. In some embodiments, the method further includes configuring the ultrasound device to collect the first and second ultrasound data at the beginning and end, respectively, of the three-dimensional ultrasound imaging sweep.

In some embodiments, the method further includes automatically performing an action based on determining that the difference between the first and second ultrasound data exceeds the threshold difference. In some embodiments, automatically performing the action comprises configuring the ultrasound device to abort the three-dimensional imaging sweep. In some embodiments, the method further includes generating a notification that the three-dimensional imaging sweep was aborted due to excessive motion. In some embodiments, performing the action comprises configuring the ultrasound device to restart the three-dimensional imaging sweep. In some embodiments, automatically performing the action comprises generating a notification that motion has occurred.

According to another aspect, a method for detecting motion during collection of ultrasound data comprises automatically determining, based on motion data from a motion sensor on an ultrasound device during the collection of the ultrasound data, that an amount of motion of the ultrasound device exceeds a threshold amount of motion; and automatically performing an action based on determining that the amount of motion of the ultrasound device exceeds the threshold amount of motion. Automatically performing the action comprises at least one of configuring the ultrasound device to abort the collection of the ultrasound data, and configuring the ultrasound device to restart the collection of the ultrasound data.

In some embodiments, the motion data comprises motion data regarding the ultrasound device. In some embodiments, the motion sensor comprises an accelerometer. In some embodiments, the motion data comprises data regarding acceleration of the ultrasound device. In some embodiments, the motion sensor comprises a gyroscope. In some embodiments, the motion data comprises data regarding angular velocity of the ultrasound device. In some embodiments, the motion sensor comprises a magnetometer. In some embodiments, the motion data comprises data regarding orientation relative to external magnetic fields.

In some embodiments, the collection of the ultrasound data comprises a three-dimensional ultrasound imaging sweep. In some embodiments, the collection of the ultrasound data comprises collection of a time-series of two-dimensional ultrasound data. In some embodiments, the method further includes configuring the ultrasound device to collect the motion data during the collection of the ultrasound data and receiving the motion data from the ultrasound device. In some embodiments, automatically performing the action comprises configuring the ultrasound device to abort the collection of the ultrasound data, and the method further includes generating a notification that the collection of the ultrasound data was aborted due to excessive motion.

Some aspects include an apparatus configured to perform the above aspects and embodiments. Some aspects include at least one non-transitory computer-readable storage medium storing processor-executable instructions that, when executed by at least one processor, cause the at least one processor to perform the above aspects and embodiments.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 is a schematic illustration of a three-dimensional imaging sweep, in accordance with certain embodiments described herein;

FIG. 2 illustrates a process for detecting motion during collection of ultrasound data during a three-dimensional imaging sweep, in accordance with certain embodiments described herein;

FIG. 3 illustrates a process for detecting motion during collection of ultrasound data, in accordance with certain embodiments described herein;

FIG. 4 illustrates a process for detecting motion during collection of ultrasound data during a three-dimensional imaging sweep, in accordance with certain embodiments described herein; and

FIG. 5 illustrates a schematic block diagram of an example ultrasound system upon which various aspects of the technology described herein may be practiced.

### DETAILED DESCRIPTION

Three-dimensional ultrasound imaging sweeps may be useful for certain applications. For example, an ultrasound imaging sweep may be used for collecting three-dimensional data for measuring the volume of an anatomical structure and/or for generating a three-dimensional visualization of an anatomical structure. An ultrasound imaging sweep may include collecting ultrasound data sets, one after another, from different sets of locations. For example, the ultrasound imaging sweep may include collecting data along multiple scanlines within a three-dimensional volume. It may be helpful to know if both the subject being imaged and the ultrasound device remained substantially motionless during the three-dimensional imaging sweep. If the subject and/or the ultrasound device were not substantially motionless during the three-dimensional imaging sweep, this may cause distortion in two-dimensional images and/or three-dimensional images generated based on the data collected during the sweep. This may also cause measurements performed based on the data collected during the sweep to be inaccurate. Determining whether the subject and/or the ultrasound device moved during the three-dimensional imaging sweep may therefore help with interpretation of images and/or measurements. The ultrasound device may be, for example, a handheld ultrasound probe or another type of ultrasound device such as a patch or pill.

The inventors have recognized that it may be possible to determine if the subject and/or the ultrasound device have moved during a three-dimensional imaging sweep by collecting ultrasound data multiple times from the same set of locations. For example, the ultrasound device may collect ultrasound data along scanline A, then collect ultrasound data along scanline B, and then collect ultrasound data along scanline A again. A processing device (e.g., a mobile phone, tablet, or laptop) in operative communication with the ultrasound device may receive and compare the two sets of ultrasound data collected along scanline A at the two different times. If the sets of ultrasound data are significantly different, this may indicate that a significant amount of motion (by the subject and/or the ultrasound device) may have occurred between collection of the first set of ultrasound data along scanline A and collection of the second set of ultrasound data along scanline A. If the processing device determines that there is a significant difference between the sets of ultrasound data, the processing device may configure the ultrasound device to abort the three-dimensional imaging sweep, to restart the three-dimensional imaging sweep, and/or to notify the user that motion has occurred and that images and/or measurements may therefore be distorted and/or inaccurate.

In some embodiments, the ultrasound device may include a motion sensor that is configured to generate motion data regarding the ultrasound device. For example, the motion sensor may include an accelerometer configured to generate data regarding acceleration of the ultrasound device, a magnetometer configured to generate data regarding orientation of the ultrasound device relative to external magnetic fields, and/or a gyroscope configured to generate data regarding angular velocity of the ultrasound device. The inventors have also recognized that it may be possible to determine if the ultrasound device has moved during collection of ultrasound data based on motion data from the motion sensor of the ultrasound. For example, data from an accelerometer in the motion sensor may indicate if acceleration of the ultrasound device occurred during collection of ultrasound data. As another example, data from a magnetometer in the motion sensor may indicate if the orientation of the ultrasound device has changed relative to external magnetic fields. As another example, data from a gyroscope in the motion sensor may indicate if angular velocity of the ultrasound device occurred during collection of ultrasound data. If the processing device determines that an amount of motion (e.g., acceleration, magnetic orientation, or angular velocity) of the ultrasound device as detected by the motion sensor exceeded a threshold amount, the processing device may configure the ultrasound device to abort the ultrasound data collection, to restart the ultrasound data collection, and/or to notify the user that motion has occurred and that images and/or measurements may therefore be distorted and/or inaccurate.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

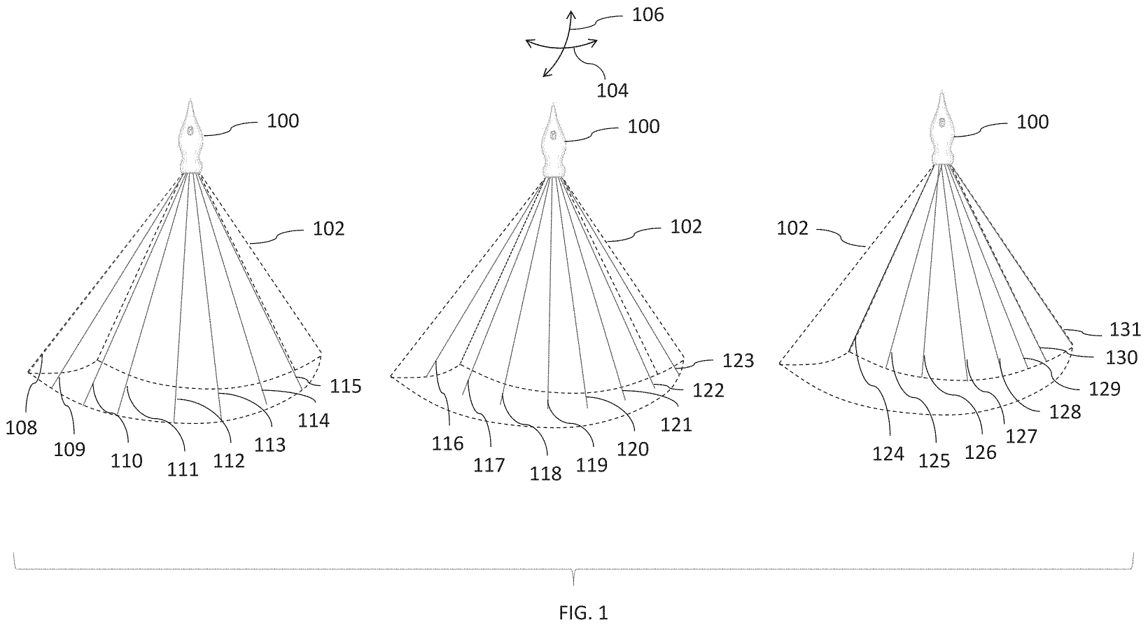

FIG. 1 is a schematic illustration of a three-dimensional imaging sweep, in accordance with certain embodiments described herein. FIG. 1 illustrates an ultrasound device 100, a three-dimensional volume 102 that is imaged during the three-dimensional imaging sweep, an azimuthal dimension 104 relative to the ultrasound device 100, and an elevational dimension 106 relative to the ultrasound device. The three-dimensional volume 110 may be within a subject. FIG. 1 further illustrates scanlines 108-131. Each of the scanlines 108-131 may include a set of points within the three-dimensional volume 102 from which the ultrasound device 100 collects ultrasound data. The scanlines 108-131 are oriented at particular angles relative to the azimuthal dimension 104 and the elevational dimension 106 of the ultrasound device 100. Certain of the scanlines 108-131 may have the same azimuthal angle and certain of the scanlines 108-131 may have the same elevational angle. For example, in FIG. 1, the scanlines 108-115 may all have the same elevational angle but each have a different azimuthal angle, the scanlines 116-123 may all have the same elevational angle but each have a different azimuthal angle, and the scanlines 124-131 may all have the same elevational angle but each have a different azimuthal angle. (It should be appreciated that the scanlines 108-131 are for illustrative purposes only, and in practice, the ultrasound device 100 may collect ultrasound data from more or fewer scanlines, more or fewer azimuthal angles, and/or more or fewer elevational angles than illustrated.) The ultrasound device 100 may collect ultrasound data along the scanlines 108-131 one after another. In some embodiments, the ultrasound device 100 may first collect data from scanlines having one elevational angle, then collect data along scanlines having another elevational angle, etc. For example, the ultrasound device 100 may first collect data from the scanline 108, then from the scanline 109, etc., up until the scanline 131. The ultrasound data collected from the scanlines 108-131 may be used to generate two-dimensional images, three-dimensional images (e.g., three-dimensional images of a fetal face), and/or to perform measurements (e.g., measurements of bladder volume).

In some embodiments, the ultrasound device 100 may remain substantially motionless while it steers ultrasound beams in different directions to collect ultrasound data along the different scanlines 108-131. The ultrasound device 100 may use a two-dimensional array of ultrasound transducers to steer the ultrasound beams in different directions (e.g., to steer the ultrasound beams at different azimuthal and elevational angles). It may be helpful to know if both the subject being imaged and the ultrasound device 100 remained substantially motionless during the three-dimensional imaging sweep. If the subject and/or the ultrasound device 100 were not substantially motionless during the three-dimensional imaging sweep, the position and/or orientation of various of the subject's anatomical structures within the three-dimensional volume 102 may change relative to the ultrasound device 100 over the course of the three-dimensional imaging sweep. This may cause distortion in two-dimensional images and/or three-dimensional images (e.g., three-dimensional images of a fetal face) generated based on the scanlines 108-131. This may also cause measurements performed based on the images (e.g., measurement of bladder volume) to be inaccurate. Determining whether the subject and/or the ultrasound device 100 moved during the three-dimensional imaging sweep may therefore help with interpretation of images and/or measurements.

The inventors have recognized that it may be possible to determine if the subject and/or the ultrasound device 100 have moved during a three-dimensional imaging sweep by collecting ultrasound data multiple times from the same scanline, known as a reference scanline. For example, the ultrasound device 100 may collect ultrasound data along scanline A, then collect ultrasound data along scanline B, and then collect ultrasound data along scanline A again. A processing device (e.g., a mobile phone, tablet, or laptop) in operative communication with the ultrasound device may be configured to receive and compare the two sets of ultrasound data collected along scanline A at the two different times. If the sets of ultrasound data are significantly different, this may indicate that a significant amount of motion (by the subject and/or the ultrasound device 100) may have occurred between collection of the first set of ultrasound data along scanline A and collection of the second set of ultrasound data along scanline A. The ultrasound device 100 may repeatedly collect ultrasound data along a particular scanline throughout the three-dimensional imaging sweep. In some embodiments, the ultrasound device 100 may interleave scans of a particular reference scanline throughout scans of the set of scanlines used for imaging. For example, if scanline 119 is the reference scanline, the ultrasound device 100 may collect ultrasound data along the scanline 119, then collect ultrasound data along the scanline 109, then collect ultrasound data along the scanline 119 again, then collect ultrasound data along the scanline 110, then collect ultrasound data along the scanline 119 again, then collect ultrasound data along the scanline 111, etc. As another example, the reference scanline may be scanned after scanning every two, three, four, etc. scanlines used for imaging. For example, the ultrasound device 100 may collect ultrasound data along the scanline 119, then collect ultrasound data along the scanlines 108-111, then collect ultrasound data along the scanline 119, then collect ultrasound data along the scanlines 112-115, then collect ultrasound data along the scanline 119 again, then collect ultrasound data along the scanlines 116-119, etc. As another example, the ultrasound device may scan the reference scanline at the beginning of the three-dimensional imaging sweep and the end of the three-dimensional imaging sweep. For example, the ultrasound device may collect ultrasound data along the scanline 119, then collect data along the scanlines 108-131, and then collect ultrasound data along the scanline 119 along. In some embodiments, the reference scanline may be the scanline at 0 elevational degrees and 0 azimuthal degrees. However, any other scanline or set of scanlines that are repeatedly imaged may be used.

It should be appreciated that a processing device (e.g., a mobile phone, tablet, or laptop) in operative with the ultrasound device may configure the ultrasound device to collect ultrasound data in any of the manners described above. The processing device may be configured to receive the sets of ultrasound data collected at multiple times from the reference scanline(s) and compare them to determine a difference between the sets of ultrasound data. In some embodiments, the processing device may be configured to compare the sets of ultrasound data by computing the cross-correlation between the sets of ultrasound data. In some embodiments, the processing device may be configured to compare the sets of ultrasound data by computing the average absolute difference between the sets of ultrasound data. For example, in the latter embodiment, computing the average absolute difference may include computing the average of the absolute differences between corresponding data points in two scanlines. In some embodiments, the processing device may be configured to compare the sets of ultrasound data by computing the maximum absolute difference between the sets of ultrasound data. For example, in the latter embodiment, computing the maximum absolute difference may include computing the maximum of the absolute differences between corresponding data points in two scanlines. In some embodiments, computing the average absolute difference and/or computing the maximum absolute difference between the first and second ultrasound data may include computing the average absolute difference and/or computing the maximum absolute difference between the first and second ultrasound data when scaled. For example, the first and second ultrasound data may be scaled by the maximum absolute value in either the first or second ultrasound data. In some embodiments, the processing device may be configured to low-pass filter the ultrasound data prior to the comparison to mitigate noise in the computed difference.

Based on comparing the sets of ultrasound data collected along the reference scanline, the processing device may determine that there is a significant difference between the sets of ultrasound data. For example, the cross-correlation between the sets of ultrasound data may be less than a threshold value, or the maximum absolute difference between the sets of ultrasound data may be greater than a threshold value, or the average absolute difference between the sets of ultrasound data may be greater than a threshold value. This may indicate that a significant amount of motion of the subject has occurred between the times when the scanline was imaged. If the processing device determines that there is a significant difference between the sets of ultrasound data, the processing device may configure the ultrasound device to an appropriate action. For example, the processing device may configure the ultrasound device abort the three-dimensional imaging sweep, to configure the ultrasound device to restart the three-dimensional imaging sweep, and/or to notify the user that motion has occurred and that images and/or measurements may therefore be distorted and/or inaccurate.

In some embodiments, the processing device may configure the ultrasound device to repeatedly collect data from multiple reference scanlines, and perform an action (e.g., configure the ultrasound device to abort the three-dimensional imaging sweep, to restart the three-dimensional imaging sweep, and/or to notify the user that motion has occurred and that images and/or measurements may therefore be distorted and/or inaccurate) based on comparing the data from the data collected from the multiple reference scanlines. For example, the processing device may perform the action if the data collected at two times from any of the reference scanlines exceeds a threshold difference, or the processing device may perform the action if the average absolute difference between all the data collected at two times from the reference scanlines exceeds a threshold difference.

While the above description of FIG. 1 has described repeatedly collecting data along a scanline, in some embodiments the processing device may configure the ultrasound device to repeatedly collect data from a location or set of locations that may not be a scanline.

It should be appreciated that the motion sensor on the ultrasound device may not be able to detect motion of the subject. Thus, embodiments using the motion sensor may be used to detect motion of the ultrasound device but not to detect motion of the subject. It should also be appreciated that a motion sensor on the ultrasound device may be used to detect motion of the ultrasound device during a three-dimensional imaging sweep as well as during collection of a time-series of two-dimensional ultrasound images. For example, embodiments using the motion sensor may be used to detect motion of the ultrasound device between collection of two two-dimensional ultrasound images of the heart during one or more heartbeats.

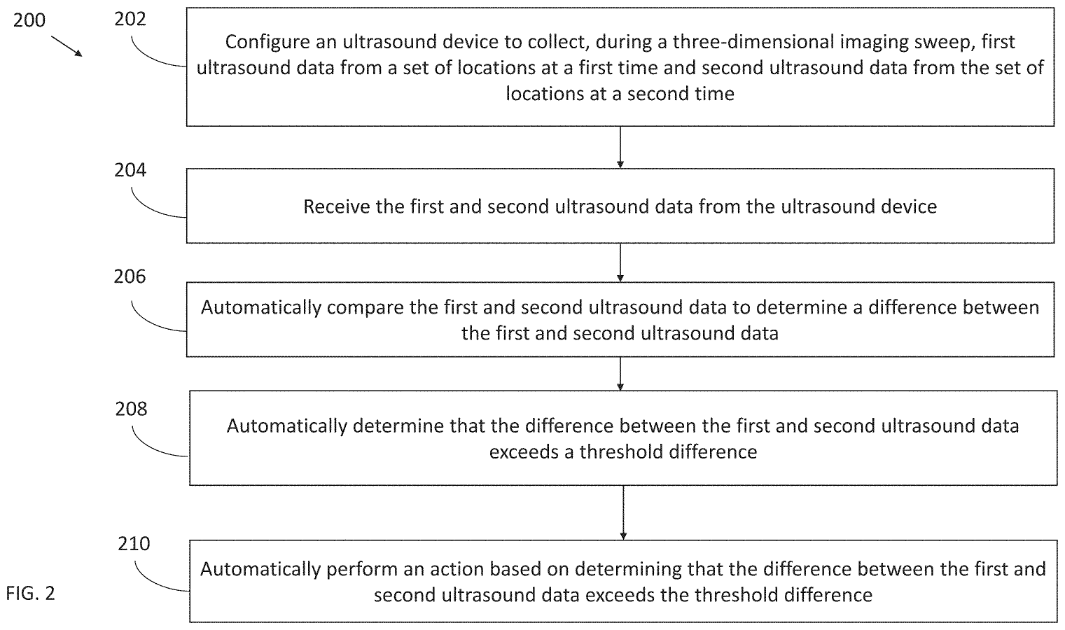

FIG. 2 illustrates a process 200 for detecting motion during collection of ultrasound data during a three-dimensional imaging sweep, in accordance with certain embodiments described herein. The process 200 is performed by a processing device in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself may perform the process 200.

In act 202, the processing device configures the ultrasound device to collect, during a three-dimensional imaging sweep, first ultrasound data from a set of locations at a first time and second ultrasound data from the same set of locations at a second time. (It should be appreciated that “first” and “second” as used with reference to FIG. 2 are used to differentiate among sets of ultrasound data and among different times, and do not necessarily imply any actual order within a group. For example, the first ultrasound data may not necessarily be the first ultrasound data collected during a particular three-dimensional imaging sweep.) In some embodiments, to configure the ultrasound device, the processing device may transmit commands to the ultrasound device over a communication link. The set of locations may be one set among multiple sets of location within a three-dimensional volume from which the ultrasound device collects ultrasound data. For example, the set of locations may be one scanline among multiple scanlines within a three-dimensional volume from which the ultrasound device collects ultrasound data. Each of the scanlines may be oriented at particular angles relative to the azimuthal dimension and the elevational dimension of the ultrasound device. The processing device may configure the ultrasound device to collect ultrasound data from each set of locations one after another. In some embodiments, the ultrasound device may remain substantially motionless while it steers ultrasound beams in different directions to collect ultrasound data from the different sets of locations. The ultrasound device 100 may use a two-dimensional array of ultrasound transducers to steer the ultrasound beams in different directions (e.g., to steer the ultrasound beams at different azimuthal and elevational angles). The first and second times are during the three-dimensional imaging sweep.

As part of act 202, the processing device configures the ultrasound device to collect ultrasound data multiple times from a particular set of locations. For example, consider an embodiment in which the processing device configures the ultrasound device to scan a particular reference scanline multiple times. In some embodiments, the processing device may configure the ultrasound device to interleave scans of the reference scanline throughout scans of the set of scanlines used for imaging. For example, the processing device may configure the ultrasound device to scan the reference scanline after scanning every scanline or every two, three, four, or any other suitable number of scanlines used for imaging. Thus, in act 202, the first and second ultrasound data may be data collected along a reference scanline at different times, and in between those times, one or more other scanlines used for imaging may be scanned. As another example, the ultrasound device may scan the reference scanline at the beginning of the three-dimensional imaging sweep and the end of the three-dimensional imaging sweep. Thus, in act 202, the first and second ultrasound data may be data collected along a reference scanline at different times, and in between those times, all scanlines used for imaging may be scanned. In some embodiments, the reference scanline may be a scanline at 0 elevational degrees and 0 azimuthal degrees. However, any other set of locations (e.g., any other scanline) that is repeatedly imaged may be used.

In some embodiments, the first and second ultrasound data may be raw acoustical data, and the processing device may configure the ultrasound device to collect the raw acoustical data. In some embodiments, the first and second ultrasound data may be scanlines, and the processing device may configure the ultrasound device to collect raw acoustical data and generate the scanlines from the raw acoustical data. In some embodiments, the first and second ultrasound data may be ultrasound images, and the processing device may configure the ultrasound device to collect raw acoustical data, generate scanlines from the raw acoustical data, and generate the ultrasound images from the scanlines. Alternatively, the processing device may configure the ultrasound device to generate the ultrasound images from the raw acoustical data without generating scanlines. In some embodiments (e.g., when an ultrasound device performs the process 200), the ultrasound device may configure itself to collect the data at act 202. The process 200 proceeds from act 202 to act 204.

In act 204, the processing device receives the first and second ultrasound data from the ultrasound device. As described above, the first and second ultrasound data may be raw acoustical data, scanlines, or ultrasound images. The processing device may receive the first and second ultrasound data from the ultrasound device over a communication link. The process 200 proceeds from act 204 to act 206.

In act 206, the processing device automatically compares the first and second ultrasound data to determine a difference between the first and second ultrasound data. In some embodiments, comparing the first and second ultrasound data may include computing a cross-correlation between the first and second ultrasound data. In some embodiments, comparing the first and second ultrasound data may include computing the average absolute difference between the first and second ultrasound data. For example, in the latter embodiment, if the first and second ultrasound data are scanlines, computing the average absolute difference may include computing the average of the absolute differences between corresponding data points in the two scanlines. In some embodiments, comparing the first and second ultrasound data may include computing the maximum absolute difference between the first and second ultrasound data. For example, in the latter embodiment, if the first and second ultrasound data are scanlines, computing the maximum absolute difference may include computing the maximum of the absolute differences between corresponding data points in the two scanlines. In some embodiments, computing the average absolute difference and/or computing the maximum absolute difference between the first and second ultrasound data may include computing the average absolute difference and/or computing the maximum absolute difference between the first and second ultrasound data when scaled. For example, the first and second ultrasound data may be scaled by the maximum absolute value in either the first or second ultrasound data. In some embodiments, the processing device may not compare the exact same data that was received at act 204. Rather, the processing device may compare data generated based on the first and second ultrasound data received at act 204. For example, the processing device may receive raw acoustical data from the ultrasound device, generate scanlines from the raw acoustical data, and compare scanlines. In some embodiments, the processing device may low-pass filter the first and second ultrasound data prior to the comparison in act 206 to mitigate noise in the computed difference. The process 200 proceeds from act 206 to act 208.

In act 208, the processing device automatically determines if the difference between the first and second ultrasound data (as computed in act 206) exceeds a threshold difference. For example, in embodiments in which comparing the first and second ultrasound data may include computing a cross-correlation between the first and second ultrasound data, determining whether the difference exceeds a threshold difference may include determining whether the cross-correlation is below a certain threshold value. In some embodiments, the threshold value may be between or equal to approximately 50%-80%. As another example, in embodiments in which comparing the first and second ultrasound data may include computing the average absolute difference between the first and second ultrasound data, determining whether the difference exceeds a threshold difference may include determining whether the average absolute difference exceeds a certain value. As another example, in embodiments in which comparing the first and second ultrasound data may include computing the maximum absolute difference between the first and second ultrasound data, determining whether the difference exceeds a threshold difference may include determining whether the maximum absolute difference exceeds a certain value. In some embodiments, the average absolute difference and/or the maximum absolute difference between the first and second ultrasound data may be the average absolute difference and/or the maximum absolute difference between the first and second ultrasound data when scaled. For example, the first and second ultrasound data may be scaled by the maximum absolute value in either the first or second ultrasound data. In such embodiments, determining whether the difference exceeds a threshold difference may include determining whether the difference exceeds a certain scaled threshold value. Also in such embodiments, the threshold value may be between or equal to approximately 50%-80%. The process 200 proceeds from act 208 to act 210.

In act 210, based on determining (in act 208) that the difference between the first and second ultrasound data exceeds a threshold difference, the processing device automatically performs an action. In some embodiments, the action may include configuring the ultrasound device to abort the ultrasound data collection. In such embodiments, the processing device may generate a notification for the user (e.g., on the display screen of the processing device) that the ultrasound data collection was aborted due to excessive motion of the subject and/or the ultrasound device. In some embodiments, the action may include configuring the ultrasound device to restart the ultrasound data collection. In some embodiments, the action may include generating a notification for the user (e.g., on the display screen of the processing device) that motion of the subject and/or the ultrasound device has occurred, and that two-dimensional images and/or three-dimensional images that are generated based on collected ultrasound data may be distorted, and/or that measurements performed based on ultrasound data collected during the ultrasound data collection may be inaccurate. In embodiments which include comparing a cross-correlation between the first and second ultrasound data to a threshold value, the processing device may generate a notification for the user if the cross-correlation is below one threshold value (e.g., a threshold value between or equal to approximately 70%-80%) and abort or restart the ultrasound data collection if the cross-correlation is below a second threshold value (e.g., a threshold value between or equal to approximately 50%-60%). In embodiments which include comparing an average absolute difference and/or a maximum absolute difference between the first and second ultrasound data when scaled (e.g., by the maximum absolute value in either set of ultrasound data) to a threshold value, the processing device may generate a notification for the user if the average absolute difference and/or a maximum absolute difference is above one threshold value (e.g., a threshold value between or equal to approximately 50%-60%) and abort or restart the ultrasound data collection if the average absolute difference and/or a maximum absolute difference is above a second threshold value (e.g., a threshold value between or equal to approximately 70%-80%).

In some embodiments, at act 202, the processing device may configure the ultrasound device to collect data from multiple sets of locations at two times. In such embodiments, at act 204, the processing device may receive the sets of locations from all the sets of locations, and automatically compare the two sets of data from each set of locations at act 206. In act 208, the processing device may automatically determine if the difference between the two sets of data from any set of locations exceeds a threshold difference and based on this determination, perform the action in 210. Alternatively, in act 208, the processing device may automatically determine if the average of the differences between the two sets of data from all the sets of locations exceeds a threshold difference and based on this determination, perform the action in 210.

In some embodiments, act 210 may be absent. For example, an action may be manually performed based on the determination in act 208, or no action may be taken. In some embodiments, act 208 may be absent. For example, the determination that the difference between the first and second ultrasound data exceeds a threshold difference may be made manually. In some embodiments, act 206 may be absent. For example, the first and second ultrasound data may be compared manually. In some embodiments, act 204 may be absent. For example, the ultrasound device may itself perform the comparison and determination at acts 206-208, and thus another device may not need to receive the ultrasound data at act 204. In some embodiments, act 202 may be absent. For example, the ultrasound device may be already configured to collect the ultrasound data in the manner of act 202.

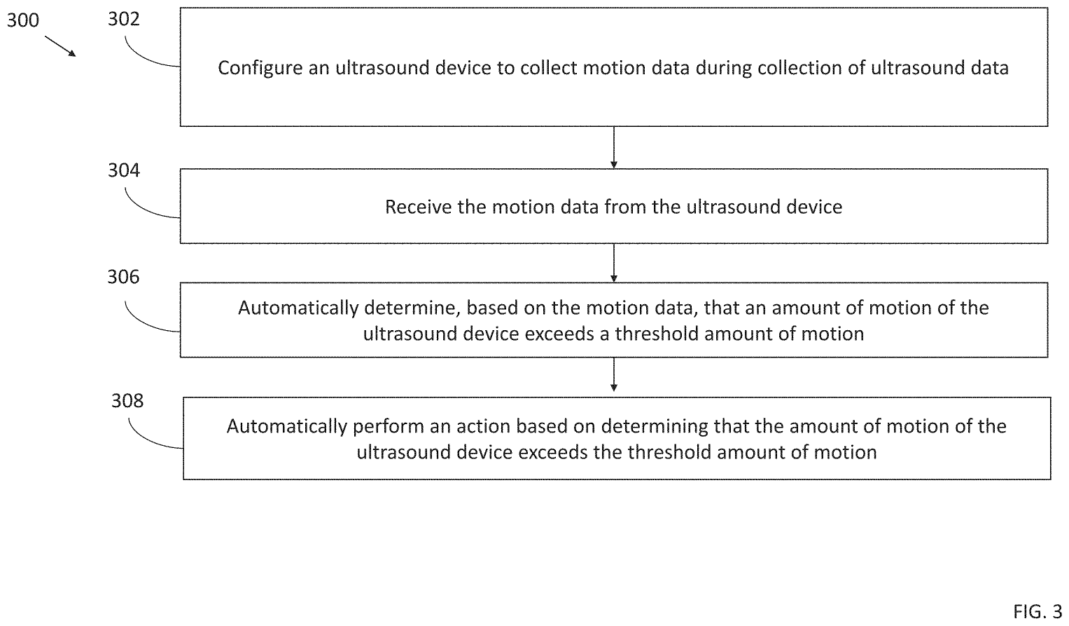

FIG. 3 illustrates a process 300 for detecting motion during collection of ultrasound data, in accordance with certain embodiments described herein. The process 300 is performed by a processing device in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself may perform the process 300.

In act 302, the processing device configures the ultrasound device to collect motion data during collection of ultrasound data. The motion data may be motion data regarding the ultrasound device. In some embodiments, the ultrasound device may include a motion sensor that is configured to generate motion data regarding the ultrasound device. For example, the motion sensor may include an accelerometer configured to generate data regarding acceleration of the ultrasound device, a magnetometer configured to generate data regarding orientation of the ultrasound device relative to external magnetic fields, and/or a gyroscope configure to generate data regarding angular velocity of the ultrasound device. The motion data may include acceleration data from an accelerometer, data regarding orientation relative to external magnetic fields from a magnetometer, and/or angular velocity data from a gyroscope that was collected during collection of the ultrasound data. The ultrasound data collection may include, for example, a three-dimensional imaging sweep or collection of a time-series of two-dimensional ultrasound data. The first and second times are during the ultrasound data collection (e.g., during the three-dimensional imaging sweep or during collection of the time-series of two-dimensional ultrasound data.) In some embodiments (e.g., when an ultrasound device performs the process 300), the ultrasound device may configure itself to collect the data at act 302. The process 300 proceeds from act 302 to act 304.

In act 304, the processing device receives the motion data from the ultrasound device. The processing device may receive the motion data from the ultrasound device over a communication link. The process 300 proceeds from act 304 to act 306.

In act 306, the processing device automatically determines, based on the motion data, that an amount of motion of the ultrasound device exceeds a threshold amount of motion. For example, the processing device may determine if the amount of linear acceleration as indicated by the motion data exceeds a threshold amount of acceleration, if the change in orientation of the ultrasound device relative to external magnetic fields exceeds a threshold change in orientation, and/or if the amount of angular velocity as indicated by the motion data exceeds a threshold amount of angular velocity. The process 300 proceeds from act 306 to act 308.

In act 308, based on determining (in act 306) that the amount of motion of the ultrasound device exceeds the threshold amount of motion, the processing device performs an action. In some embodiments, the action may include configuring the ultrasound device to abort the ultrasound data collection. In such embodiments, the processing device may generate a notification for the user (e.g., text on the display screen of the processing device, graphics on the display screen of the processing screen, and/or audio outputted by a speaker of the processing device) that the ultrasound data collection was aborted due to excessive motion of the subject and/or the ultrasound device. In some embodiments, the action may include configuring the ultrasound device to restart the ultrasound data collection. In some embodiments, the action may include generating a notification for the user (e.g., text on the display screen of the processing device, graphics on the display screen of the processing screen, and/or audio outputted by a speaker of the processing device) that motion of the subject and/or the ultrasound device has occurred, and that images that are generated based on collected ultrasound may be distorted, and/or that measurements performed based on collected ultrasound data may be inaccurate.

In some embodiments, act 308 may be absent. For example, an action may be manually performed based on the determination in act 306, or no action may be taken. In some embodiments, act 306 may be absent. For example, the determination that the amount of motion exceeds the threshold may be performed manually. In some embodiments, act 304 may be absent. For example, the ultrasound device may itself perform the comparison and determination at acts 306-308, and thus another device may not need to receive the ultrasound data at act 304. In some embodiments, act 302 may be absent. For example, the ultrasound device may be already configured to collect the motion data in the manner of act 302.

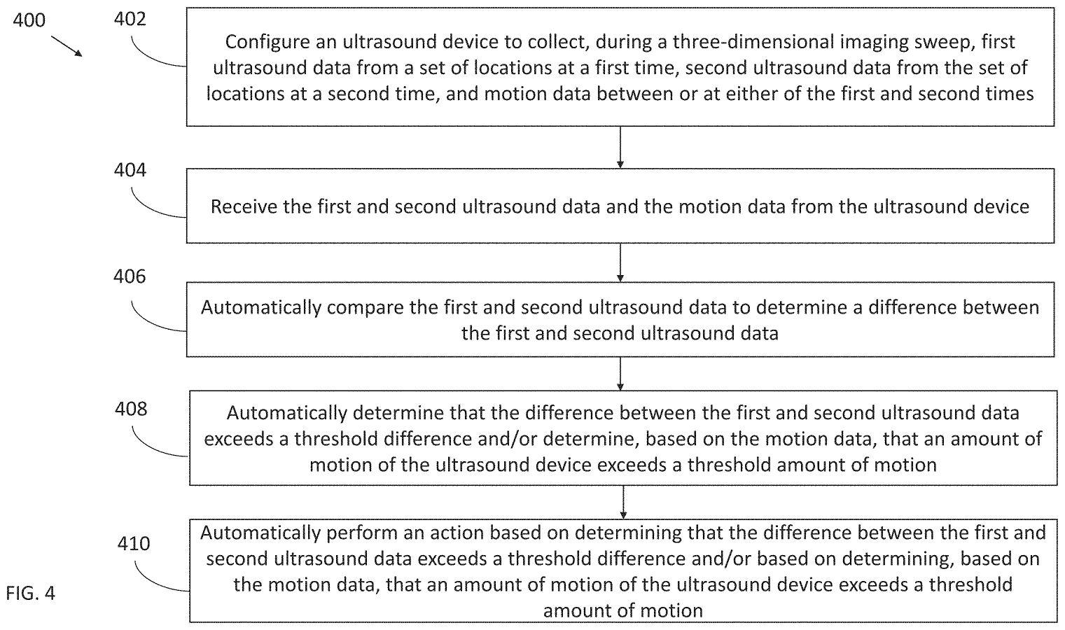

FIG. 4 illustrates a process 400 for detecting motion during collection of ultrasound data during a three-dimensional imaging sweep, in accordance with certain embodiments described herein. The process 400 is performed by a processing device in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself may perform the process 400.

In act 402, the processing device configures the ultrasound device to collect, during the three-dimensional imaging sweep, first ultrasound data from a set of locations at a first time, second ultrasound data from the set of locations at a second time, and motion data between or at either of the first and second times. Further description of collecting ultrasound data at first and second times may be found with reference to act 202. Further description of collecting motion data may be found with reference to act 302. In some embodiments (e.g., when an ultrasound device performs the process 400), the ultrasound device may configure itself to collect the data at act 402. The process 400 proceeds from act 402 to act 404.

In act 404, the processing device receives the first and second ultrasound data and the motion data from the ultrasound device. Further description of receiving ultrasound and motion data may be found with reference to acts 204 and 304. The process 400 proceeds from act 404 to act 406.

In act 406, the processing device automatically compares the first and second ultrasound data to determine a difference between the first and second ultrasound data. Further description of comparing ultrasound data may be found with reference to act 206. The process 400 proceeds from act 406 to act 408.

In act 408, the processing device automatically determines that the difference between the first and second ultrasound data exceeds a threshold difference and/or determines, based on the motion data, that an amount of motion of the ultrasound device exceeds a threshold amount of motion. Further description of automatically determining that a difference between ultrasound data exceeds a threshold difference may be found with reference to act 208. Further description of determining based on motion data that an amount of motion of the ultrasound device exceeds a threshold amount of motion may be found with reference to act 306. The process 400 proceeds from act 408 to act 410.

In act 410, the processing device automatically performs an action based on determining that the difference between the first and second ultrasound data exceeds a threshold difference and/or based on determining, based on the motion data, that an amount of motion of the ultrasound device exceeds a threshold amount of motion. Further description of automatically performing an action may be found with reference to act 410 and act 308. In some embodiments, the processing device may automatically perform the action based on determining both that the difference between the first and second ultrasound data exceeds the threshold difference and that the amount of motion of the ultrasound device exceeds the threshold amount of motion. In some embodiments, the processing device may automatically perform the action based on determining either that the difference between the first and second ultrasound data exceeds the threshold difference or that the amount of motion of the ultrasound device exceeds the threshold amount of motion.

In some embodiments, act 410 may be absent. For example, an action may be manually performed based on the determination in act 408, or no action may be taken. In some embodiments, act 408 may be absent. For example, the determination that the difference between the first and second ultrasound data exceeds a threshold difference and/or the determination that the amount of motion exceeds the threshold amount of motion may be made manually. In some embodiments, act 406 may be absent. For example, the first and second ultrasound data may be compared manually. In some embodiments, act 404 may be absent. For example, the ultrasound device may itself perform the comparison and determination at acts 406-408, and thus another device may not need to receive the ultrasound and motion data at act 404. In some embodiments, act 402 may be absent. For example, the ultrasound device may be already configured to collect the ultrasound and motion data in the manner of act 402.

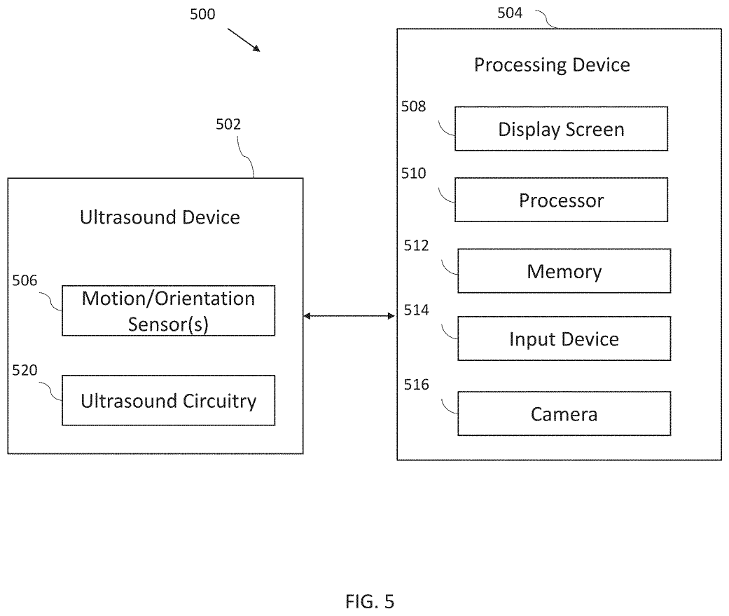

FIG. 5 illustrates a schematic block diagram of an example ultrasound system 500 upon which various aspects of the technology described herein may be practiced. The ultrasound system 500 includes an ultrasound device 502 and a processing device 504. The ultrasound device 502 may be the same as the ultrasound device 100 and/or the ultrasound device discussed with reference to the processes 200-400. The processing device 504 may be the same as the processing device discusses with reference to FIG. 1 and the processes 200-400.

The ultrasound device 502 includes a motion and/or orientation sensor(s) 506 and ultrasound circuitry 520. The processing device 504 includes a camera 516, a display screen 508, a processor 510, a memory 512, and an input device 514. The processing device 504 is in wired (e.g., through a lightning connector or a mini-USB connector) and/or wireless communication (e.g., using BLUETOOTH, ZIGBEE, and/or WiFi wireless protocols) with the ultrasound device 502.

The ultrasound device 502 may be configured to generate ultrasound data that may be employed to generate an ultrasound image. The ultrasound device 502 may be constructed in any of a variety of ways. In some embodiments, the ultrasound device 502 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 520 may be configured to generate the ultrasound data. The ultrasound circuitry 520 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 520 (e.g., transmit circuitry, receive circuitry, control circuitry, power management circuitry, and processing circuitry) to form a monolithic ultrasound device. The ultrasound device 502 may transmit ultrasound data and/or ultrasound images to the processing device 504 over a wired (e.g., through a lightning connector or a mini-USB connector) and/or wireless (e.g., using BLUETOOTH, ZIGBEE, and/or WiFi wireless protocols) communication link.

The motion and/or orientation sensor(s) 506 may be configured to generate motion and/or orientation data regarding the ultrasound device 502. For example, the motion and/or orientation sensor(s) 506 may be configured to generate data regarding acceleration of the ultrasound device 502, data regarding angular velocity of the ultrasound device 502, and/or data regarding magnetic force acting on the ultrasound device 502 due to the local magnetic field, which in many cases is simply the field of the earth. The motion and/or orientation sensor(s) 506 may include an accelerometer, a gyroscope, and/or a magnetometer. Depending on the sensors present in the motion and/or orientation sensor(s) 506, the motion and/or orientation data generated by the motion and/or orientation sensor(s) 506 may describe three degrees of freedom, six degrees of freedom, or nine degrees of freedom for the ultrasound device 502. For example, the motion and/or orientation sensor(s) 506 may include an accelerometer, a gyroscope, and/or magnetometer. Each of these types of sensors may describe three degrees of freedom. If the motion and/or orientation sensor(s) 506 includes one of these sensors, the motion and/or orientation sensor(s) 506 may describe three degrees of freedom. If the motion and/or orientation sensor(s) 506 includes two of these sensors, the motion and/or orientation sensor(s) 506 may describe two degrees of freedom. If the motion and/or orientation sensor(s) 506 includes three of these sensors, the motion and/or orientation sensor(s) 506 may describe nine degrees of freedom. The ultrasound device 502 may transmit data to the processing device 504 over a wired (e.g., through a lightning connector or a mini-USB connector) and/or wireless (e.g., using BLUETOOTH, ZIGBEE, and/or WiFi wireless protocols) communication link.

Referring now to the processing device 504, the processor 510 may include specially-programmed and/or special-purpose hardware such as an application-specific integrated circuit (ASIC). For example, the processor 510 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network. The processing device 504 may be configured to process the ultrasound data received from the ultrasound device 502 to generate ultrasound images for display on the display screen 508. The processing may be performed by, for example, the processor 510. The processor 510 may also be adapted to control the acquisition of ultrasound data with the ultrasound device 502. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing device 504 may be configured to perform certain of the processes described herein using the processor 510 (e.g., one or more computer hardware processors) and one or more articles of manufacture that include non-transitory computer-readable storage media such as the memory 512. The processor 510 may control writing data to and reading data from the memory 512 in any suitable manner. To perform certain of the processes described herein, the processor 510 may execute one or more processor-executable instructions stored in one or more non-transitory computer-readable storage media (e.g., the memory 512), which may serve as non-transitory computer-readable storage media storing processor-executable instructions for execution by the processor 510. The camera 516 may be configured to detect light (e.g., visible light) to form an image or a video. The display screen 508 may be configured to display images and/or videos, and may be, for example, a liquid crystal display (LCD), a plasma display, and/or an organic light emitting diode (OLED) display on the processing device 504. The input device 514 may include one or more devices capable of receiving input from a user and transmitting the input to the processor 510. For example, the input device 514 may include a keyboard, a mouse, a microphone, touch-enabled sensors on the display screen 508, and/or a microphone. The display screen 508, the input device 514, the camera 516, and the speaker 506 may be communicatively coupled to the processor 510 and/or under the control of the processor 510.

It should be appreciated that the processing device 504 may be implemented in any of a variety of ways. For example, the processing device 504 may be implemented as a handheld device such as a mobile smartphone or a tablet. Thereby, a user of the ultrasound device 502 may be able to operate the ultrasound device 502 with one hand and hold the processing device 504 with another hand. In other examples, the processing device 504 may be implemented as a portable device that is not a handheld device, such as a laptop. In yet other examples, the processing device 504 may be implemented as a stationary device such as a desktop computer. For further description of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application) and published as U.S. Patent Pub. 2017-0360397 A1, which is incorporated by reference herein in its entirety.

FIG. 5 should be understood to be non-limiting. For example, the ultrasound device 502 and/or the processing device 504 may include fewer or more components than shown. As a particular example, the motion and/or orientation sensor(s) 506 may not be necessary for performing the process 200.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Various inventive concepts may be embodied as one or more processes, of which an example has been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus for detecting motion during a three-dimensional ultrasound imaging sweep, the apparatus comprising:
a processing device in operative communication with an ultrasound device, the processing device configured to automatically compare first ultrasound data collected from a set of locations at a first time by the ultrasound device and second ultrasound data collected from the set of locations at a second time by the ultrasound device, wherein the first and second times are during the three-dimensional ultrasound imaging sweep.

2. The apparatus of claim 1, wherein the set of locations comprises a scanline.

3. The apparatus of claim 1, wherein the ultrasound device is configured to remain substantially motionless during the three-dimensional ultrasound imaging sweep.

4. The apparatus of claim 1, wherein the set of locations is one set among multiple sets of location within a three-dimensional volume from which the ultrasound device collects ultrasound data during the three-dimensional ultrasound imaging sweep.

5. The apparatus of claim 1, wherein the processing device is configured, when automatically comparing the first and second ultrasound data, to determine a difference between the first and second ultrasound data by:
computing a cross-correlation between the first and second ultrasound data;
computing a maximum absolute difference between the first and second ultrasound data; or
computing an average absolute difference between the first and second ultrasound data.

6. The apparatus of claim 5, wherein the processing device is further configured to:
automatically determine that the difference between the first and second ultrasound data exceeds a threshold difference.

7. The apparatus of claim 6, wherein the processing device is further configured to:
automatically perform an action based on determining that the difference between the first and second ultrasound data exceeds the threshold difference.

8. The apparatus of claim 7, wherein the processing device is configured, when automatically performing the action, to configure the ultrasound device to abort the three-dimensional imaging sweep.

9. The apparatus of claim 8, wherein the processing device is further configured to generate a notification that the three-dimensional imaging sweep was aborted due to excessive motion.

10. The apparatus of claim 7, wherein the processing device is configured, when automatically performing the action, to configure the ultrasound device to restart the three-dimensional imaging sweep.

11. The apparatus of claim 7, wherein the processing device is configured, when automatically performing the action, to generate a notification that motion has occurred.

12. The apparatus of claim 6, wherein the processing device is configured:
when automatically comparing the first and second ultrasound data to determine the difference between the first and second ultrasound data by computing a cross-correlation between the first and second ultrasound data, and when automatically determining that the difference between the first and second ultrasound data exceeds a threshold difference, to determine that the cross-correlation is less than a certain value;
when automatically comparing the first and second ultrasound data to determine the difference between the first and second ultrasound data by computing a maximum absolute difference between the first and second ultrasound data, and when automatically determining that the difference between the first and second ultrasound data exceeds a threshold difference, to determine that the maximum absolute difference is greater than a certain value; or
when automatically comparing the first and second ultrasound data to determine the difference between the first and second ultrasound data by computing an average absolute difference between the first and second ultrasound data, and when automatically determining that the difference between the first and second ultrasound data exceeds a threshold difference, to determine that the average absolute difference is greater than a certain value.

13. The apparatus of claim 1, wherein the processing device is further configured to:
configure the ultrasound device to collect the first ultrasound data from the set of locations at the first time and the second ultrasound data from the set of locations at the second time; and
receive the first and second ultrasound data.

14. The apparatus of claim 13, wherein the processing device is further configured to configure the ultrasound device to collect ultrasound data from only one other set of locations between the first time and the second time.

15. The apparatus of claim 13, wherein the processing device is further configured to configure the ultrasound device to collect ultrasound data from multiple other set of locations between the first time and the second time.

16. The apparatus of claim 13, wherein the processing device is further configured to configure the ultrasound device to collect the first and second ultrasound data at a beginning and end, respectively, of the three-dimensional ultrasound imaging sweep.

17. An apparatus for detecting motion during collection of ultrasound data, the apparatus comprising:
a processing device in operative communication with an ultrasound device, the processing device configured to:
automatically determine, based on motion data from a motion sensor on the ultrasound device during the collection of the ultrasound data, that an amount of motion of the ultrasound device exceeds a threshold amount of motion; and
automatically perform an action based on determining that the amount of motion of the ultrasound device exceeds the threshold amount of motion, wherein automatically performing the action comprises at least one of:
configuring the ultrasound device to abort the collection of the ultrasound data; or
configuring the ultrasound device to restart the collection of the ultrasound data.

18. The apparatus of claim 17, wherein the motion data comprises motion data regarding the ultrasound device.

19. The apparatus of claim 17, wherein the collection of the ultrasound data comprises a three-dimensional ultrasound imaging sweep.

20. The apparatus of claim 17, wherein the collection of the ultrasound data comprises collection of a time-series of two-dimensional ultrasound data.

21. The apparatus of claim 17, wherein the processing device is further configured to:
configure the ultrasound device to collect the motion data during the collection of the ultrasound data; and
receive the motion data from the ultrasound device.

22. The apparatus of claim 17, wherein the processing device is configured, when automatically performing the action, to configure the ultrasound device to abort the collection of the ultrasound data, and the processing device is further configured to generate a notification that the collection of the ultrasound data was aborted due to excessive motion.
